(19) United States
(12) Patent Application Publication
(10) Pub. No.: US 2020/0330203 A1
(43) Pub. Date: Oct. 22, 2020

(54) HEMOSTATIC PASTE WITH LIGHT-CURABLE FEATURE

(71) Applicant: Norman F. Dahl, III, Branford, CT (US)

(72) Inventor: Norman F. Dahl, III, Branford, CT (US)

(21) Appl. No.: 16/850,602

(22) Filed: Apr. 16, 2020

Related U.S. Application Data
(60) Provisional application No. 62/835,100, filed on Apr. 17, 2019.

(51) Int. Cl. A61C 13/15; A61C 19/06; A61K 31/724; A61K 47/10; A61K 47/46; A61K 47/32; A61K 47/02; A61K 9/06; A61K 9/00

(52) U.S. Cl. CPC A61C 19/004; A61C 19/063; A61K 31/724; A61K 47/10; A61K 9/0063; A61K 47/32; A61K 47/02; A61K 9/06; A61K 47/46

(57) ABSTRACT

A hemostatic paste comprising: a hemostatic component; and a light-curable component; wherein the hemostatic component comprises (2-hydroxypropyl)-Y-cyclodextrin.

### REFERENCE TO PENDING PRIOR PATENT APPLICATION

This patent application claims benefit of pending prior U.S. Provisional Patent Application Ser. No. 62/835,100, filed Apr. 17, 2019 by Norman F. Dahl for HEMOSTATIC PASTE WITH LIGHT-CURABLE FEATURE (Attorney's Docket No. DAHL-2 PROV), which patent application is hereby incorporated herein by reference.

### FIELD OF THE INVENTION

This invention relates to hemostatic compounds in general, and more particularly to hemostatic compounds for use in dental procedures.

### BACKGROUND OF THE INVENTION

Open wounds bleed and, if left untreated, can become infected. Wounds are often treated (i.e., closed) using simple “first aid” articles (e.g., bandages, gauze, etc.) or, if the wound is larger and/or deeper, by applying sutures and/or staples to close the open wound.

However, in some instances a wound is intentional and cannot be closed immediately. By way of example but not limitation, during many surgical procedures, incisions must be made which bleed, causing blood loss to the patient and obscuring the surgical field. Sponges, gauze, cotton batting, etc. are sometimes used to absorb blood during surgery and/or to promote blood clotting. However, such sponges, gauze, cotton batting, etc. may not be appropriate in some circumstances (e.g., the sponges, gauze, cotton batting, etc. may be too large for the incision, they may obscure the surgical field, they may need to be held in place during the procedure, etc.).

Because sponges, gauze, cotton batting, etc. may not be appropriate for many surgical procedures, hemostatic compounds are sometimes used to promote blood clotting around an incision (or wound) and thereby reduce bleeding.

By way of example but not limitation, hemostatic compounds are sometimes used in dental procedures to temporarily stop bleeding during a procedure or during the time between procedures. By way of further example but not limitation, hemostatic compounds are sometimes used during procedures for cementing dental crowns. In such procedures, the gum is typically retracted from the tooth, causing trauma to the gum (gingiva) and resulting in bleeding. Hemostatic compounds may be used by the dentist to inhibit such bleeding from the gum (gingiva).

While such hemostatic compounds are generally effective in reducing bleeding from the gum (gingiva), the viscosity of the hemostatic compound may be too low for the hemostatic compound to stay properly positioned on the gum (gingiva) for a desired period of time. In other words, the hemostatic compound may not have a viscosity high enough to keep the hemostatic compound from flowing away from its initial deployment site.

Thus there is a need for a new and improved hemostatic compound which can stay properly positioned on the gum (gingiva) for a desired period of time.

### SUMMARY OF THE INVENTION

The present invention comprises the provision and use of a novel hemostatic compound which is engineered to stay properly positioned on the gum (gingiva) for a desired period of time.

In one preferred form of the invention, the novel hemostatic compound comprises a novel hemostatic paste which is configured to stay properly positioned on the gum (gingiva) for a desired period of time.

In one particularly preferred form of the invention, the novel hemostatic compound comprises a novel hemostatic paste which is light-curable so as to allow the novel hemostatic paste to be selectively hardened upon demand.

Among other things, the novel hemostatic compound is particularly well suited for use in procedures for mounting dental crowns, and also to stop sulcular bleeding during restorative procedures, wherein the hemostatic compound may be deployed as a paste around the tooth at the gum-line so as to staunch bleeding, and light-cured in-situ into a semi-hardened structure that is pliable so as to serve as a hemostatic barrier for holding the gum (gingiva) away from the tooth and maintain control of bleeding while exposing the base sub-gingival aspects of a tooth. The semi-hardened hemostatic compound may thereafter be removed from the site by teasing the semi-hardened structure away from the tissue prior to taking a dental impression, or placing a final restoration.

The novel hemostatic compound of the present invention may also be used in many other types of dental restorative procedures where hemostasis needs to be achieved prior to final restorative placement.

In one form of the invention, there is provided a hemostatic paste comprising:

a hemostatic component; and

a light-curable component;

wherein the hemostatic component comprises (2-hydroxypropyl)-Y-cyclodextrin.

In another form of the invention, there is provided a method for preparing a light-curable hemostatic paste, the method comprising:

preparing a light-curable component;

preparing a hemostatic component, wherein the hemostatic component comprises (2-hydroxypropyl)-Y-cyclodextrin; and

mixing the light-curable component and the hemostatic component together so as to form the hemostatic paste.

In another form of the invention, there is provided a method for providing hemostasis to a wound, the method comprising:

using a deployment device to deliver a hemostatic paste to a wound, wherein the hemostatic paste comprises a hemostatic component and a light-curable component, and wherein the hemostatic component comprises (2-hydroxypropyl)-Y-cyclodextrin.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIG. 1 is a schematic view showing the novel hemostatic paste as it emerges from a syringe;

FIG. 2 is a schematic view showing how the novel hemostatic paste may be smeared after it has emerged from a syringe and before it is cured;

FIG. 3 is a schematic view showing the novel hemostatic paste being cured with light;

FIG. 4 is a schematic view showing the novel hemostatic paste after curing; and

FIGS. 5-16 show an exemplary dental crown procedure utilizing the novel hemostatic paste of the present invention.

### DETAILED DESCRIPTION OF THE PREFERRED EMBODIMENTS

The present invention comprises the provision and use of a novel hemostatic compound which is engineered to stay properly positioned on the gum (gingiva) for a desired period of time.

In one preferred form of the invention, the novel hemostatic compound comprises a novel hemostatic paste which is configured to stay properly positioned on the gum (gingiva) for a desired period of time.

In one particularly preferred form of the invention, the novel hemostatic compound comprises a novel hemostatic paste which is light-curable so as to allow the novel hemostatic paste to be selectively hardened upon demand.

Among other things, the novel hemostatic compound is particularly well suited for use in procedures for cementing dental crowns, and also to stop sulcular bleeding during restorative procedures, wherein the hemostatic compound may be deployed as a paste around the tooth at the gum-line so as to staunch bleeding, and light-cured in-situ into a semi-hardened structure so as to serve as a hemostatic barrier for holding the gum (gingiva) away from the tooth and maintain control of bleeding while exposing the base sub-gingival aspects of a tooth. The semi-hardened hemostatic compound may thereafter be removed from the site by teasing the semi-hardened structure away from the tissue prior to taking a dental impression, or placing a final restoration.

The novel hemostatic compound of the present invention may also be used in many other types of dental restorative procedures where hemostasis needs to be achieved prior to final restorative placement.

### The Novel Hemostatic Paste in General

More particularly, in one form of the present invention, there is provided a novel hemostatic paste comprising a hemostatic component and a light-curable component (sometimes hereinafter referred to as a “photo-initiator”) which are combined together so as to form a hemostatic paste which is light-curable.

In one preferred form of the invention, the hemostatic component of the novel hemostatic paste comprises (2-hydroxypropyl)-Y-cyclodextrin (99.8% pure) which is suspended in a viscous, non-toxic polyol compound, e.g., glycerin.

And in one possible preferred form of the invention, the hemostatic compound comprises a flavoring agent, e.g., honey, a strawberry extract, a mint extract, etc.

In one form of the invention, the light-curable component comprises methacrylate ester monomers in a light-cured, glass-filled paste.

In one form of the invention, the light-curable component comprises a commercially-available product such as Pulpdent Kool-Dam™ (Pulpdent Corporation of Watertown, Mass.) or a similar commercially-available product.

In one form of the invention, the light-curable component comprises a proprietary formulation comprising Ethoxylated bis phenol A dimethacrylate Mw 1700, Urethane dimethacrylate, Amorphous Glass (silanized), Ethyl 4-dimethylaminobenzoate, Camphorquinone (initiator), Ethylene glycol dimethacrylate, p-Methoxyphenol, Glycol methacrylate, Butylated Hydroxytoluene, Titanium Dioxide Pigment and Blue Pigment Ci 42090.

Preferably the light-curable component is curable using light which is generally just above the UV spectrum. In one preferred form of the invention, the light-curable component may be cured with a light-emitting device having an output peak wavelength range of 450 nm-470 nm, and an output intensity from 1100 mW/cm² to 1330 mW/cm².

In accordance with the present invention, the hemostatic component and the light-curable component are blended together so as to provide a paste having hemostatic properties. The hemostatic paste has a viscosity which is low enough to be flowable but high enough that the hemostatic paste does not flow under the influence of gravity alone. Significantly, the hemostatic paste may be selectively semi-hardened upon demand by the application of light of an appropriate wavelength. Also significantly, the uncured hemostatic paste is slightly tacky to help it stay in place, but the semi-hardened hemostatic paste does not adhere to a tooth, bone or tissue.

### First Preferred Formulation

In one form of the invention, the novel hemostatic paste comprises the formulation shown in Table 1 below:

TABLE 1

| | |
|---|---|
| Light-Curable Component | |
| Pulpdent Kool-Dam ™ | 60 Wt. % |
| Hemostatic Component | |
| (2-hydroxypropyl)-Y-cyclodextrin | 40 Wt. % |
| (molecular weight = 1,580) sterile, 99.8% pure | |
| Total | 100 Wt. % |

This formulation provides a novel hemostatic paste which is configured to stay properly positioned on the gum (gingiva) for a desired period of time. Furthermore, this formulation provides a novel hemostatic paste which is light-curable so as to allow the novel hemostatic paste to be selectively semi-hardened (e.g., to varying degrees of hardness) upon demand.

It should be appreciated that the foregoing formulation can be adjusted as desired so as to provide a novel hemostatic paste which is more or less viscous in its uncured state.

### Second Preferred Formulation

In another form of the invention, the novel hemostatic compound comprises the formulation shown in Table 2 below:

TABLE 2

| Chemical Name | CAS-No | ≈ Wt. % | |
|---|---|---|---|
| Light-Curable Component | | | |
| Ethoxylated bis phenol A | 41637-38-1 | 32.57 | Wt. % |
| dimethacrylate Mw 1700 | | | |
| Urethane dimethacrylate | 72869-86-4 | 18.85 | Wt. % |
| Amorphous Glass (silanized) | | 6.85 | Wt. % |
| Ethyl 4-dimethylaminobenzoate | 10287-53-3 | 0.34 | Wt. % |
| Camphorquinone (initiator) | 10373-78-1 | 0.34 | Wt. % |
| Ethylene glycol dimethacrylate | 97-90-5 | 0.34 | Wt. % |
| p-Methoxyphenol | 150-76-5 | 0.05 | Wt. % |
| Glycol methacrylate | 868-77-9 | 0.05 | Wt. % |
| Butylated Hydroxytoluene | 128-37-0 | 0.05 | Wt. % |
| Titanium Dioxide Pigment | 13463-67-7 | 0.34 | Wt. % |
| Blue Pigment Ci 42090 | 3844-45-9 | 0.05 | Wt. % |
| Hemostatic Component | | | |
| (2-hydroxypropyl)-Y-cyclodextrin | | 40 | Wt. % |
| Total | | 100 | Wt. % |

This formulation provides a novel hemostatic paste which is configured to stay properly positioned on the gum (gingiva) for a desired period of time. Furthermore, this formulation provides a novel hemostatic paste which is light-curable so as to allow the novel hemostatic paste to be selectively hardened (e.g., to varying degrees of hardness) upon demand.

### Third Preferred Formulation

In another form of the invention, the novel hemostatic paste comprises the formulation shown in Table 3 below:

TABLE 3

| Chemical Name | CAS-No | ≈ Wt. % | |
|---|---|---|---|
| Light-Curable Component | | | |
| Ethoxylated bis phenol | 41637-38-1 | 32.00 | Wt. % |
| A dimethacrylate Mw 1700 | | | |
| Urethane dimethacrylate | 72869-86-4 | 18.66 | Wt. % |
| Amorphous Glass (silanized) | | 7.73 | Wt. % |
| Ethyl 4-dimethylaminobenzoate | 10287-53-3 | 0.30 | Wt. % |
| Camphorquinone (initiator) | 10373-78-1 | 0.30 | Wt. % |
| Ethylene glycol dimethacrylate | 97-90-5 | 0.30 | Wt. % |
| p-Methoxyphenol | 150-76-5 | 0.06 | Wt. % |
| Glycol methacrylate | 868-77-9 | 0.06 | Wt. % |
| Butylated Hydroxytoluene | 128-37-0 | 0.06 | Wt. % |
| Titanium Dioxide Pigment | 13463-67-7 | 0.30 | Wt. % |
| Blue Pigment Ci 42090 | 3844-45-9 | 0.06 | Wt. % |
| Hemostatic Component | | | |
| (2-hydroxypropyl)-Y-cyclodextrin | | 40 | Wt. % |
| Total | | 100 | Wt. % |

This formulation provides a novel hemostatic paste which is configured to stay properly positioned on the gum (gingiva) for a desired period of time. Furthermore, this formulation provides a novel hemostatic paste which is light-curable so as to allow the novel hemostatic paste to be selectively hardened (e.g., to varying degrees of hardness) upon demand.

### Method of Manufacture

In one preferred form of the invention, the novel hemostatic paste is manufactured in the following manner.

First the light-curable component is prepared (if the light-curable component is not a commercially-available product such as Pulpdent Kool-Dam™). This is done while working in a low light environment (e.g., an environment devoid of the light wavelengths which can cure the novel hemostatic paste). The “smallest” contributors to the light-curable component (i.e., those elements of the compound having the lowest Wt %) are mixed together, i.e., the low viscosity liquids are mixed together and the granular materials are pulverized and then mixed together with the low viscosity liquids at room temperature. As additional constituents are added, the viscosity of the compound increases. Powders are sifted into the mixture while mixing in order to minimize clumping.

After the light-curable component has been prepared, the hemostatic component (2-hydroxypropyl)-Y-cyclodextrin is prepared.

Finally, the light-curable component and the hemostatic component are mixed together so as to complete the manufacture of the novel hemostatic paste.

### Delivery and Light Activation

The novel hemostatic paste may be delivered to a remote site (e.g., to a wound, to an incision, to a bleeding sulcus, etc.) by various means.

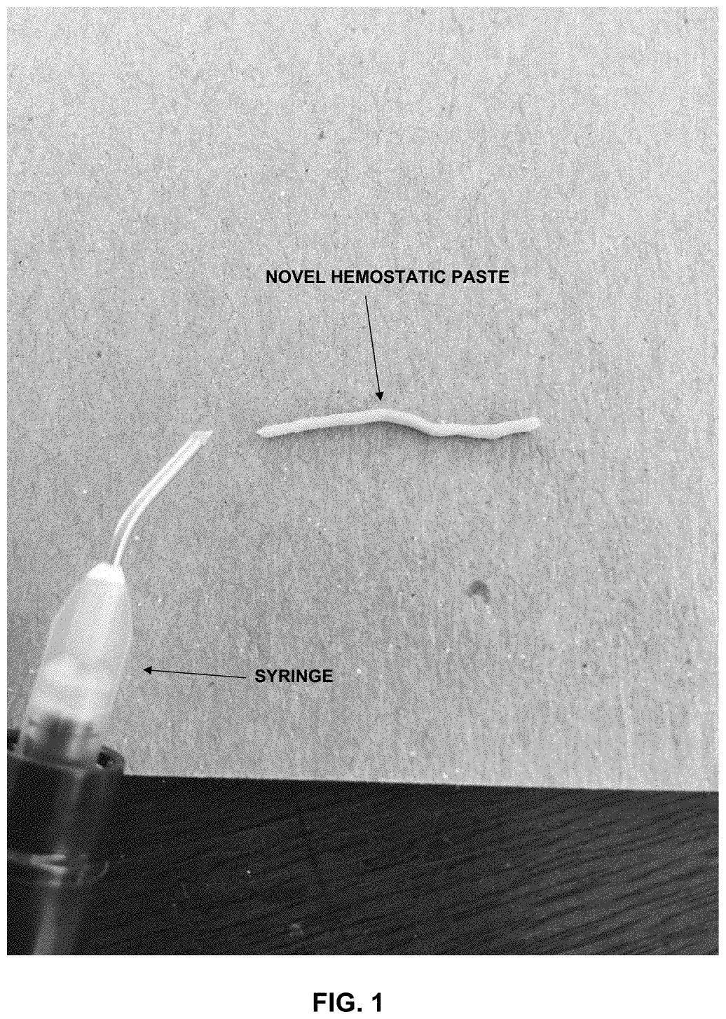

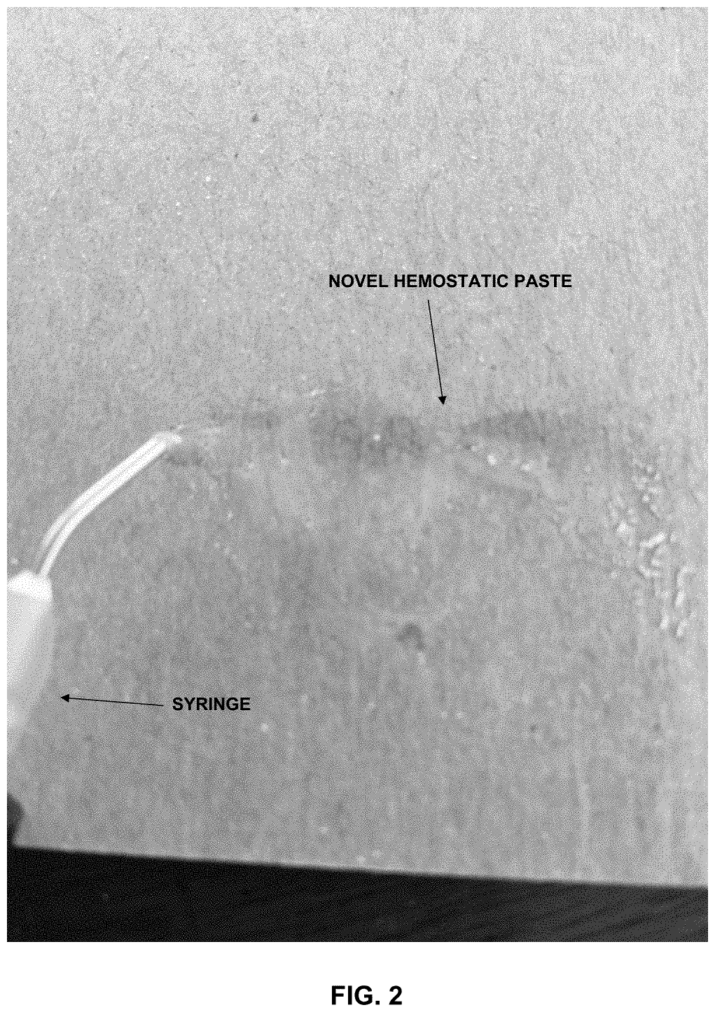

By way of example but not limitation, and looking now at FIG. 1, the novel hemostatic paste may be delivered via a syringe. Note that when the novel hemostatic paste is delivered to the operative site, it is flowable (e.g., it is relatively viscous, having a consistency like window putty, toothpaste, etc.) and has sufficient consistency to maintain its shape against moderate forces. However, and as seen in FIG. 2, the novel hemostatic paste may be easily spread (e.g., into a thin layer) if it is subjected to modest forces prior to light curing. This feature can be advantageous where it is desired to shape the novel hemostatic paste with an instrument (or a finger) at the operative site.

If desired, the tip of the syringe may be curved, angled, etc. so as to assist in deploying the novel hemostatic paste in a confined space (e.g., around a tooth within the mouth).

By way of further example but not limitation, the novel hemostatic paste of the present invention may be delivered via an ampoule (e.g., a disposable ampoule made out of low density polyethylene). Again, if desired, the tip of the ampoule may be curved, angled, etc. so as to assist in deploying the novel hemostatic paste in a confined space (e.g., within the mouth).

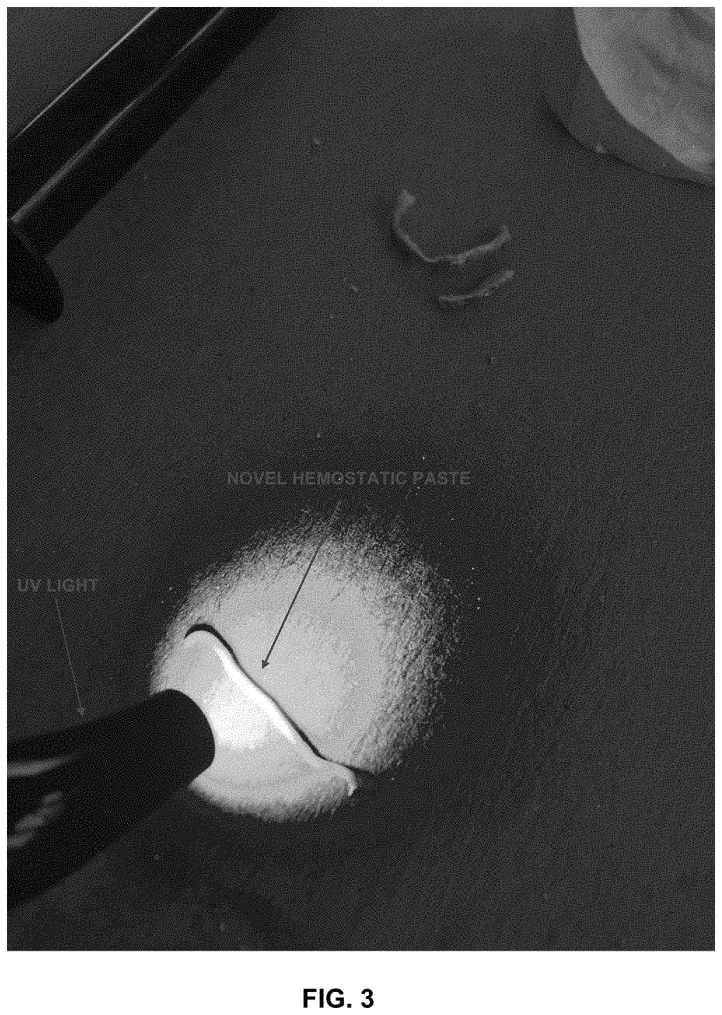

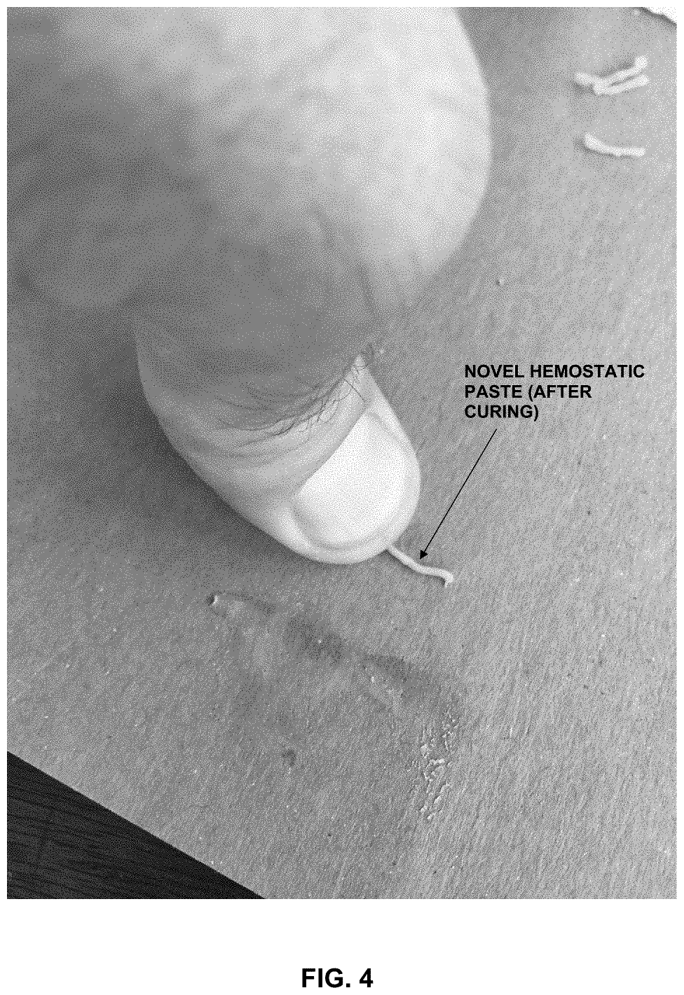

In addition to delivering the novel hemostatic paste to the desired site, the present invention also comprises the provision and use of an appropriate light source for activating the light-curable component of the novel hemostatic paste. More particularly, and looking now at FIG. 3, there is shown an exemplary light source for selectively activating (i.e., selectively curing) the light-curable component of the novel hemostatic paste of the present invention. When the light source is used to selectively activate the light-curable component, the novel hemostatic paste is selectively semi-hardened so as to be capable of withstanding sulcular forces. In this respect it should be appreciated that greater or lesser amounts of the appropriate wavelength light may be used to provide greater or lesser hardening of the novel hemostatic paste. By way of example but not limitation, the novel hemostatic paste may be cured so as to take on a consistency similar to that of an eraser. See, for example, FIG. 4. If desired, the light source may comprise a curved, angled, etc. tip for delivering the appropriate wavelength light into a confined space (e.g., around a tooth within the mouth).

If desired, the novel hemostatic paste of the present invention may be provided in the form of a kit which comprises the novel hemostatic paste pre-loaded into a deployment device (e.g., syringe, ampoule, etc.). The light source is a device capable of providing light at a wavelength appropriate to cure the novel hemostatic paste and is typically readily available in substantially all dental offices and hence it is anticipated that it will not be packaged in the kit.

### Method of Use—Coagulation Only

It should be appreciated that the light-curable component of the novel hemostatic paste may be omitted, or may remain unactivated, without compromising the utility of the hemostatic component of the novel hemostatic paste. Put another way, the novel hemostatic paste may be used to coagulate a wound even if the light-curable component is omitted from the hemostatic paste, or, if the light-curable component is present in the novel hemostatic paste, without activating the light-curable component of the novel hemostatic paste.

In this form of the present invention, the novel hemostatic paste is applied to the wound using a deployment device (e.g., a syringe, ampoule, etc.) and then the novel hemostatic compound acts to coagulate blood and reduce (or halt) bleeding.

### Method of Use—Coagulation and Solidification

Where the novel hemostatic paste of the present invention includes the light-curable component, and where the light-curable component is activated (e.g., via exposure to light of an appropriate wavelength), the novel hemostatic paste may be selectively semi-hardened (e.g., to varying degrees of hardness) upon demand.

In use, the novel hemostatic paste of the present invention is typically applied to the site of the wound (or incision, bleeding, etc.) using a deployment device (e.g., syringe, ampoule, etc.). Upon application to the tissue, the novel hemostatic paste will begin to provide hemostatic benefits. The novel hemostatic paste is then exposed to light of an appropriate wavelength in order to cure the light-curable component of the novel hemostatic paste. The appropriate wavelength light is delivered for a selected amount of time in order to cure (i.e., harden) the novel hemostatic paste to the desired degree of hardness.

### Use of the Novel Hemostatic Paste in an Exemplary Dental Crown Procedure

The novel hemostatic paste may be applied around the perimeter of a tooth during a dental procedure so as to staunch bleeding and/or to allow the semi-hardened hemostatic paste to be used as a hemostatic barrier for holding the gum (gingiva) away from the tooth so as to expose the sub-gingival aspects of a tooth.

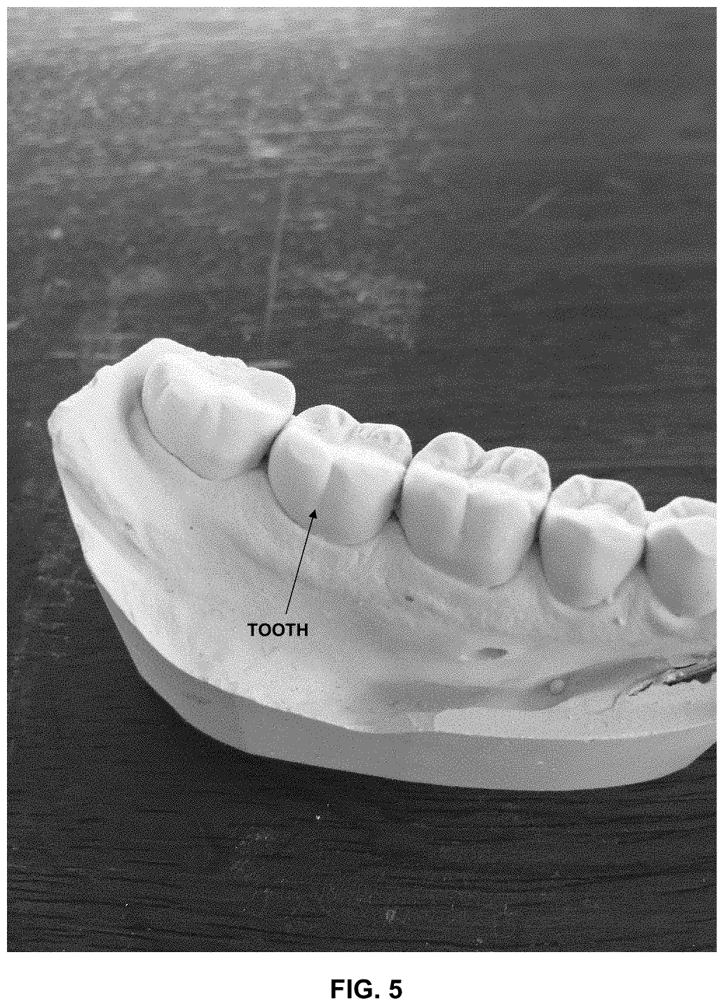

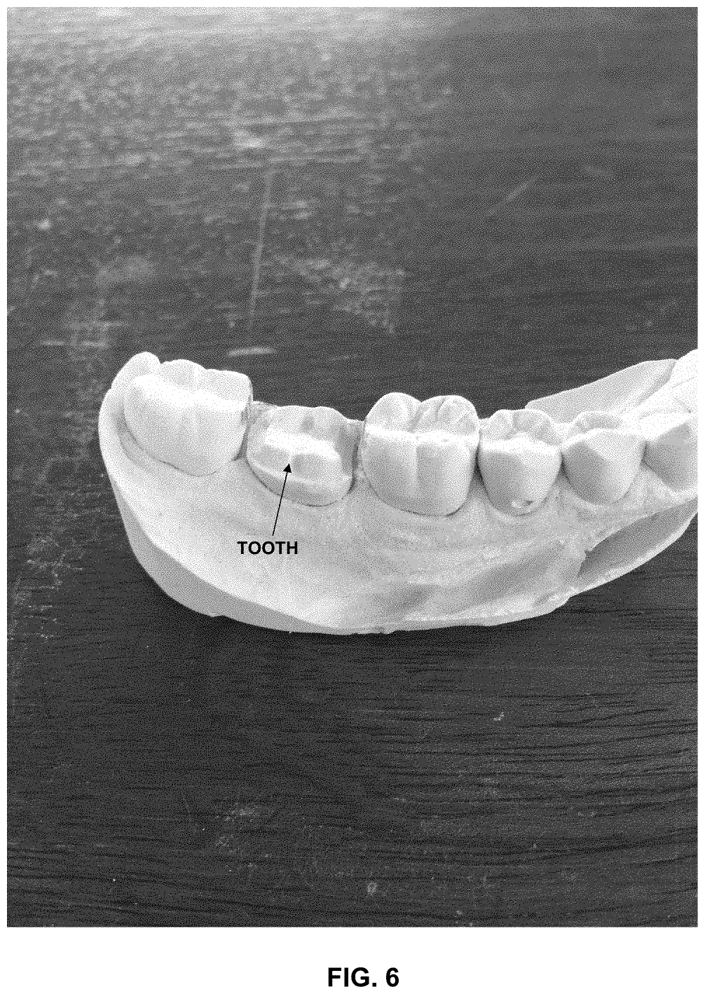

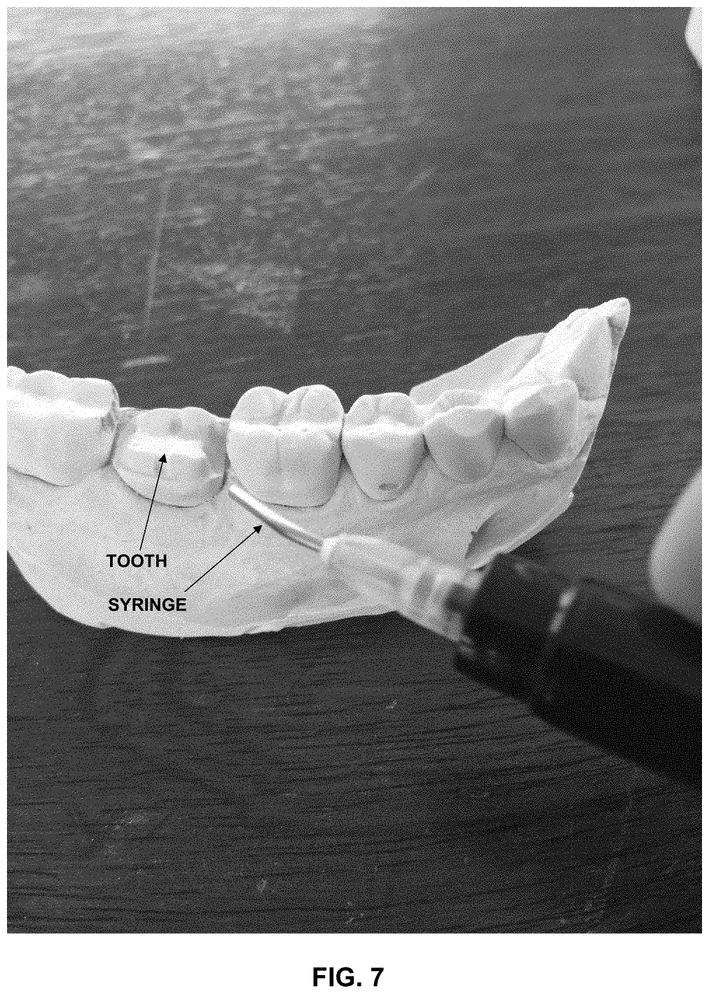

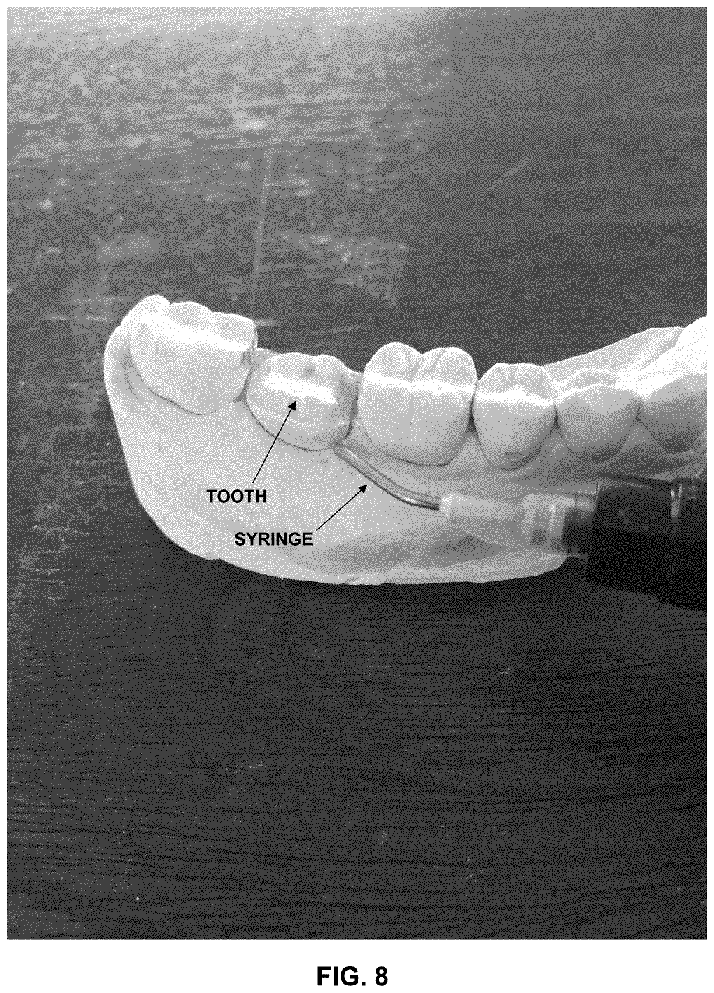

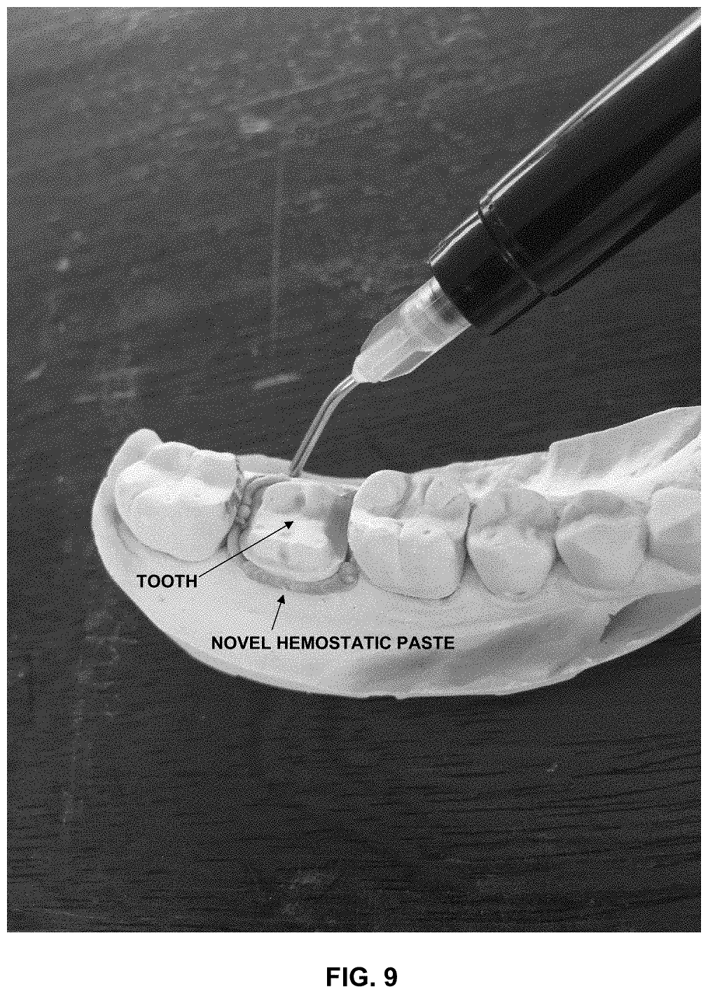

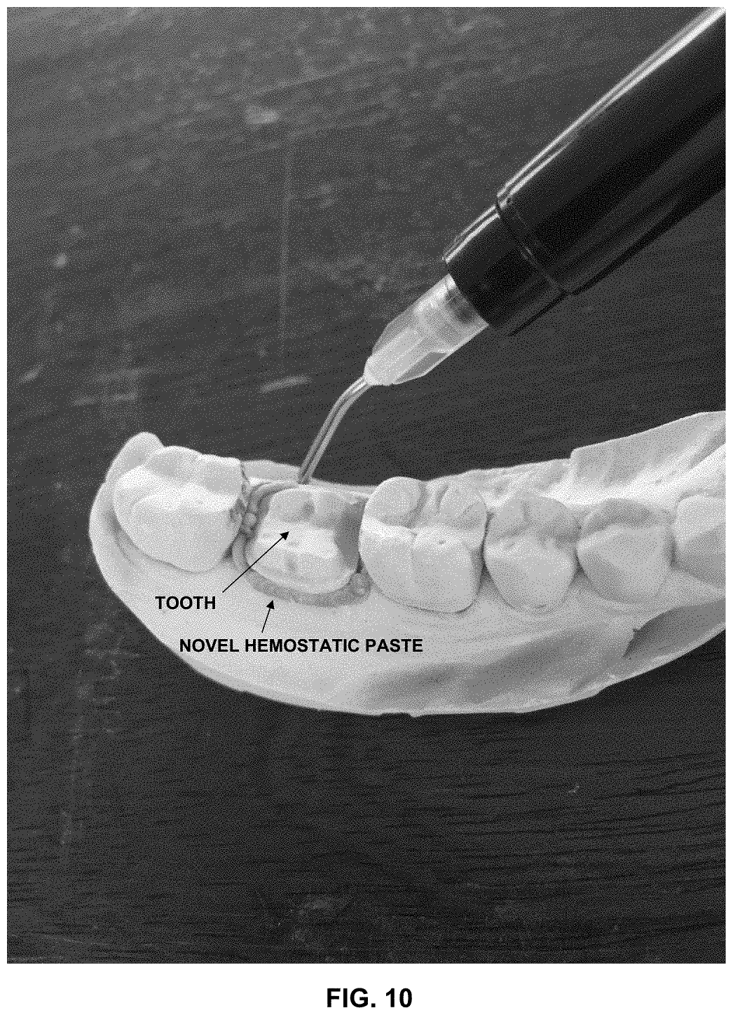

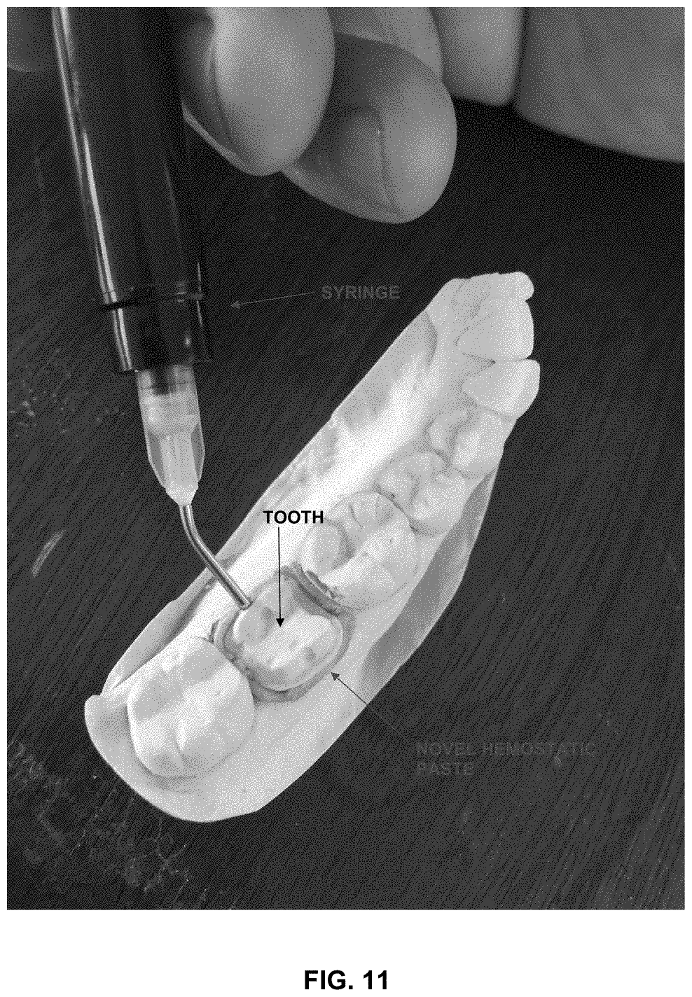

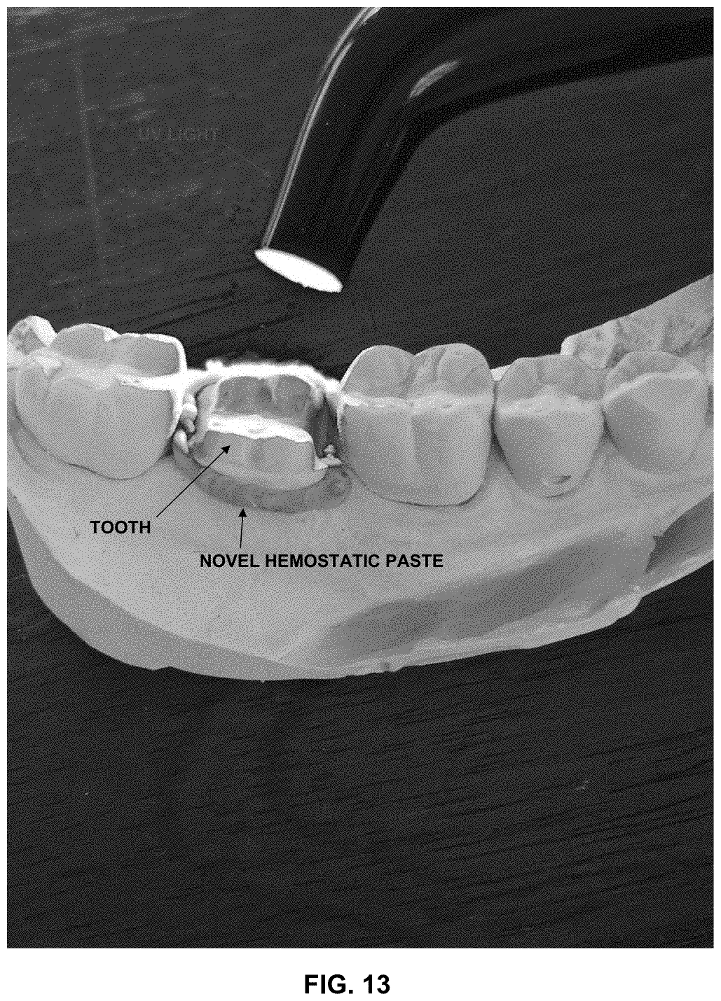

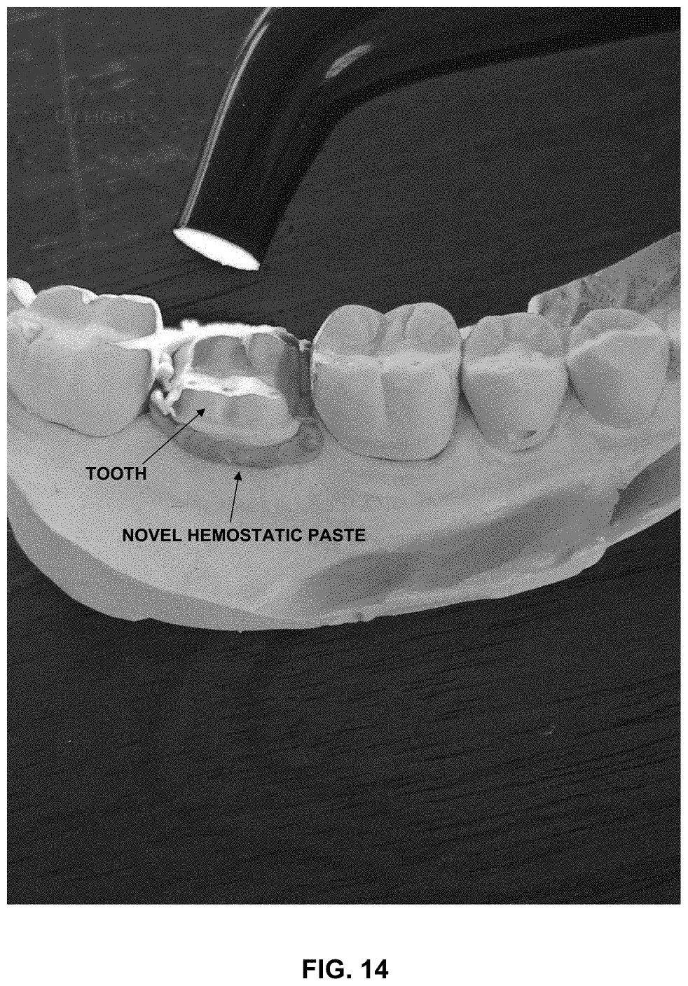

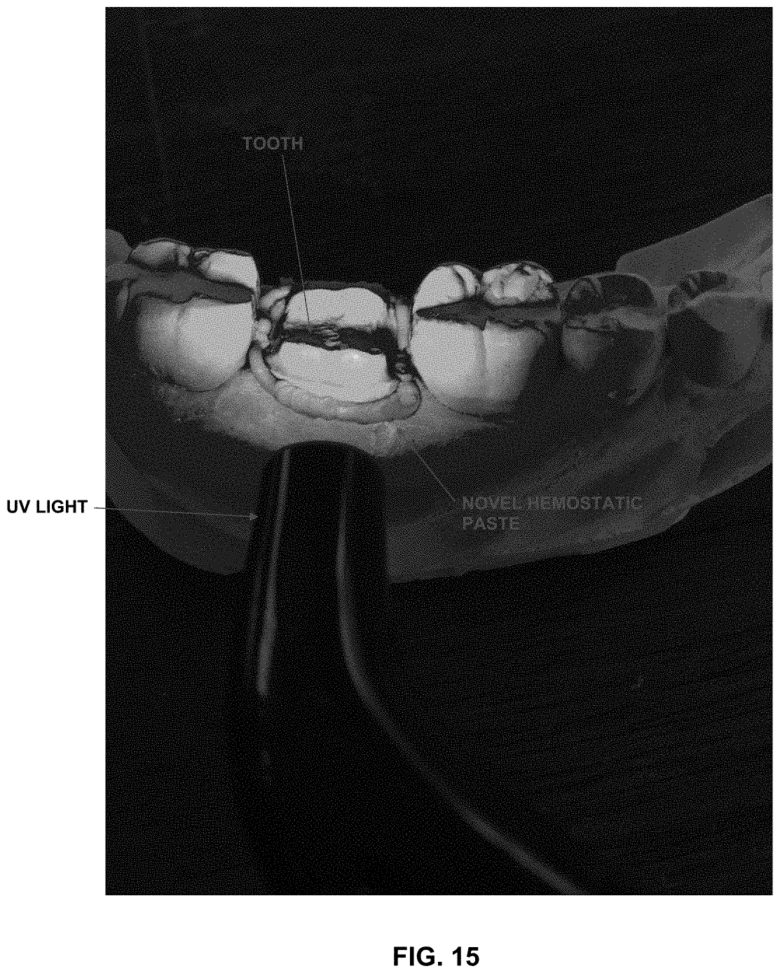

By way of example but not limitation, and looking now at FIGS. 5-16, the novel hemostatic paste may be advantageously used when cementing a dental crown to a tooth. More particularly, the tooth may first be shaped (FIGS. 5 and 6) in order to prepare the tooth to receive a dental crown. To this end, it is generally desirable for the dentist to retract the gum (gingiva) so as to provide access to the sub-gingival aspects of the tooth. However, inflammation around the temporary crown typically causes the gum (gingiva) to bleed. To reduce this bleeding, the novel hemostatic paste can be applied around the sub-gingival aspects of the tooth using a syringe (FIGS. 7-11). As the novel hemostatic paste is applied to the gum (gingiva), its hemostatic properties act to reduce bleeding from the gum (gingiva).

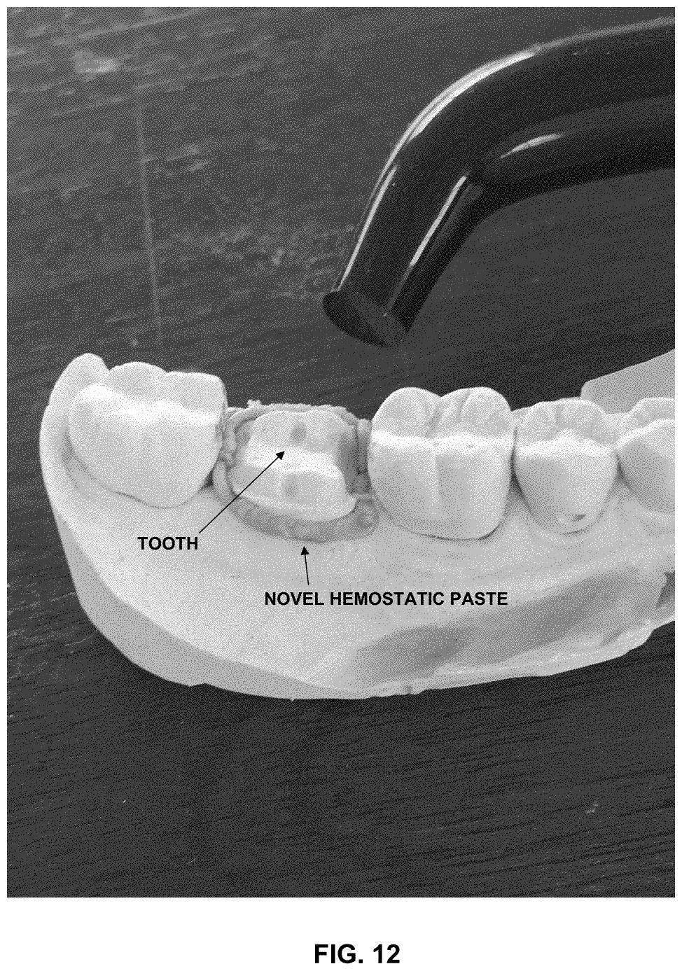

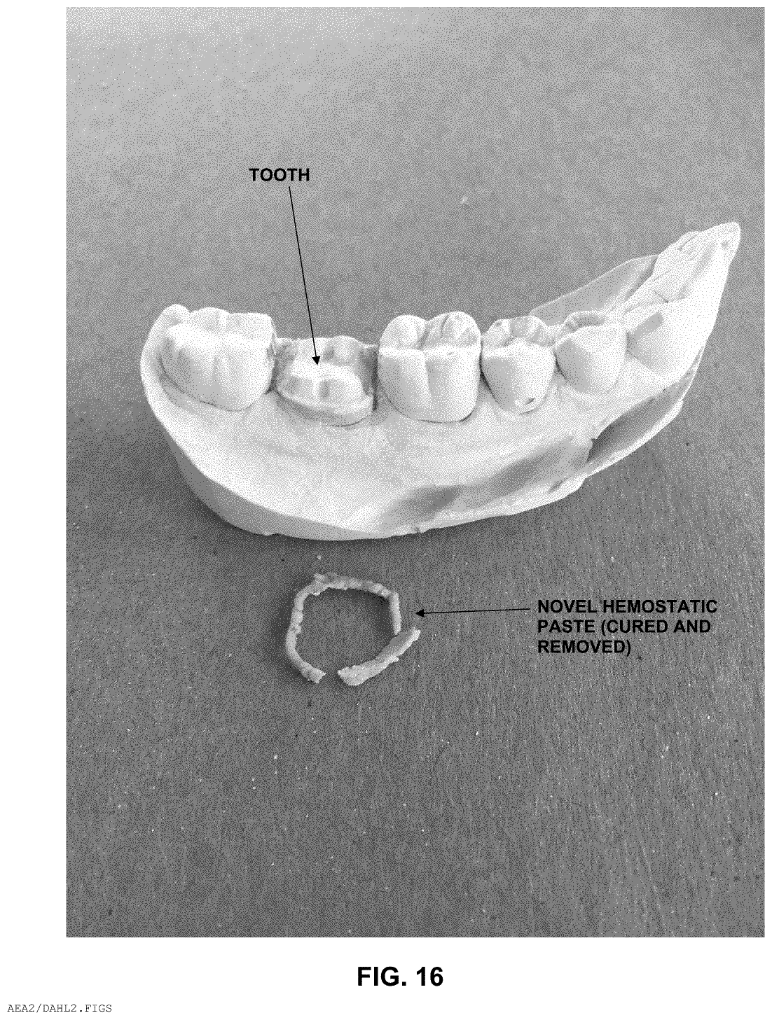

Significantly, after the novel hemostatic paste has been deployed around the perimeter of the tooth, light of the appropriate wavelength may be used to selectively harden the novel hemostatic paste (FIGS. 12-15). The light source is left in place as long as is needed in order to achieve the desired degree of hardening of the novel hemostatic paste. When the novel hemostatic paste is so hardened, the novel hemostatic paste can then also serve as a hemostatic barrier to keep the gum (gingiva) retracted from the tooth (while still providing the aforementioned hemostatic benefits). At the appropriate time, the dentist can remove the novel hemostatic paste by simply teasing the semi-hardened hemostatic paste off of the tooth (e.g., with tweezers). Significantly, while the uncured hemostatic paste is slightly tacky to help it stay in place, the semi-hardened hemostatic paste does not adhere to the tooth, bone or tissue. The novel hemostatic paste may come off as a single piece or as several large pieces. This makes it easy to “clean up” the surgical field. See FIG. 16.

It should also be appreciated that curing the light-curable element of novel hemostatic paste may help the novel hemostatic paste to better seat in the surrounding anatomy (such as the gum (gingiva) adjacent to a tooth, etc.). Curing can also make the novel hemostatic paste more resilient to various degradation elements present in the mouth (e.g., saliva).

### Other Exemplary Uses of the Novel Hemostatic Paste

It should be appreciated that the novel hemostatic paste of the present invention may be used in a wide range of applications. By way of example but not limitation, the novel hemostatic paste may be used to control bleeding during the installation of temporary or permanent prosthetics (e.g., dental crowns, inlays, bridges, etc.), to control periodontal bleeding, to control gingival bleeding during restorative procedures, post-surgically to control bleeding, to control bleeding during the taking of an impression (e.g., as a retraction paste) or placing a final restoration, to pack a bleeding cavity after a tooth extraction, or cured and used as a damn.

### MODIFICATIONS OF THE PREFERRED EMBODIMENTS

It should be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. A hemostatic paste comprising:
a hemostatic component; and
a light-curable component;
wherein the hemostatic component comprises (2-hydroxypropyl)-Y-cyclodextrin.

2. A hemostatic paste according to claim 1 wherein the hemostatic component is suspended in a non-toxic polyol compound.

3. A hemostatic paste according to claim 2 wherein the non-toxic polyol is glycerin.

4. A hemostatic paste according to claim 1 wherein the hemostatic component further comprises a flavoring agent.

5. A hemostatic paste according to claim 4 wherein the flavoring agent comprises at least one selected from the group consisting of honey, a strawberry extract and a mint extract.

6. A hemostatic paste according to claim 1 wherein the light-curable component comprises methacrylate ester monomers in a light-cured glass-filled paste.

7. A hemostatic paste according to claim 1 wherein the light-curable component comprises Ethoxylated bis phenol A dimethacrylate Mw 1700, Urethane dimethacrylate, Amorphous Glass (silanized), Ethyl 4-dimethylaminobenzoate, Camphorquinone (initiator), Ethylene glycol dimethacrylate, p-Methoxyphenol, Glycol methacrylate, Butylated Hydroxytoluene, Titanium Dioxide Pigment and Blue Pigment Ci 42090.

8. A hemostatic paste according to claim 1 wherein the light-curable component is curable with a light-emitting device having an output peak wavelength range of 450 nm to 470 nm and an output intensity from 1100 mW/cm² to 1330 mW/cm².

9. A hemostatic paste according to claim 1 wherein the hemostatic paste has a viscosity which is low enough to be flowable but high enough that the hemostatic paste does not flow by gravity alone.

10. A method for preparing a light-curable hemostatic paste, the method comprising:
preparing a light-curable component;
preparing a hemostatic component, wherein the hemostatic component comprises (2-hydroxypropyl)-Y-cyclodextrin; and
mixing the light-curable component and the hemostatic component together so as to form the hemostatic paste.

11. A method according to claim 10 wherein the light-curable component is prepared while working in an environment devoid of the light wavelengths which can cure the light-curable component.

12. A method according to claim 10 wherein the light-curable component is prepared by mixing low viscosity liquids together, pulverizing granular materials and mixing the pulverized granular materials with the mixed low viscosity liquids.

13. A method according to claim 12 wherein mixing is performed at room temperature.

14. A method according to claim 10 wherein the hemostatic component is prepared by mixing glycerin and (2-hydroxypropyl)-Y-cyclodextrin together.

15. A method according to claim 14 further comprising adding a flavoring agent to the hemostatic component.

16. A method for providing hemostasis to a wound, the method comprising:
using a deployment device to deliver a hemostatic paste to a wound, wherein the hemostatic paste comprises a hemostatic component and a light-curable component, and wherein the hemostatic component comprises (2-hydroxypropyl)-Y-cyclodextrin.

17. A method according to claim 16 wherein the deployment device comprises one of a syringe and an ampoule.

18. A method according to claim 16 further comprising activating the light-curable component so as to harden the hemostatic paste.

19. A method according to claim 18 wherein activating the light-curable component comprises exposing the hemostatic paste to light of an appropriate wavelength to harden the light-curable component of the hemostatic paste.

20. A method according to claim 16 wherein the hemostatic paste is applied around a tooth.
